(19) United States
(12) Patent Application Publication
(10) Pub. No.: US 2006/0014274 A1
(43) Pub. Date: Jan. 19, 2006

(54) Fiber cassette and modularly designed cassette system

(75) Inventor: Uwe Klaus, Radeberg (DE)

Correspondence Address: GUDRUN E. HUCKETT DRAUDT, LONSSTR. 53, WUPPERTAL, 42289, DE

(73) Assignee: Saxonia Bio Tec GmbH, Radeberg (DE)

(21) Appl. No.: 10/526,439

(22) PCT Filed: Sep. 9, 2003
(86) PCT No.: PCT/DE03/03066

§ 371 Date: Mar. 3, 2005

(51) Int. Cl. C12M 1/12

(52) U.S. Cl. 435/297.4; 210/321.8

(57) ABSTRACT

A fiber cassette has a housing that is delimited by two congruent base surfaces and at least one circumferential surface and has an interior having at least one cavity. At least one layer of fibers is arranged in the interior of the housing essentially parallel to at least one center plane of the housing, wherein ends of the fibers are anchored fixedly in the interior of the housing. A first one of the at least one cavity defines an outer compartment that surrounds the fibers externally. The at least one center plane does not intersect the base surfaces within the outer compartment. The fibers are arranged U-shaped or essentially parallel to one another and end within the interior of the housing. The housing has at least one opening for supplying and/or removing fluids.

The invention relates to a fiber cassette for use in filtration, diffusion, elimination or adsorption of fluids or substances or use as a bioreactor. Depending on the application, different shapes and materials can be used as fibers, primarily in the form of hollow fibers. Moreover, the invention relates to a cassette system that is modularly constructed of individual cassettes.

Such fiber cassettes or cassette systems are of special interest for multiple applications in chemistry, pharmacy, medicine, cellular biology, microbiology, the food industry, engineering, or biotechnology.

The term fluids is to be understood as gases, gas mixtures, as well as, generally, liquids, for example, clear solutions, protein solutions, emulsions or suspensions.

Hollow fiber modules that are used for filtration, separation, adsorption or for bioreactors are constructed according to the prior generally in tubular form. They are comprised in general of a tube and a hollow fiber type but can also comprise separate tubes and different fiber types (EP 0515034, EP 0514021, EP 0530670, EP 0285812, DE 3636583, DE 3839567, DE 3805414, DE 3423258, DE 3039336, DE 2825065, DE 2828549, EP 0282355, DE 3435883, EP 0414525, DE 3423258). These modules are identical to or derived from the configurations and fiber types as they are used in dialysis, hemofiltration or oxygenation. Accordingly, they are not especially optimized for use in connection with filtration, separation, or adsorption purposes or the area of bioreactors but only for the use in dialysis or hemofiltration or oxygenation.

The tubular configuration of the prior art systems is characterized in that their height is longer than the other dimensions and the fibers are arranged parallel to the vertical line.

As a result of the derivation from the tubular configuration—generally employed in medical technology—significant disadvantages result for many applications and in regard to flexibility of the product. For example, a combination of different hollow fiber membrane types is possible only by employing a housing specially created for the application or by external connection by means of a hose system. The latter connection has the great disadvantage that no general, large surface area, spatially proximal connection between the compartments is present, and flexibility in regard to the application is therefore lacking.

In addition to the conventional tubular bioreactors there exist also other housing shapes that operate partially with crossed hollow fibers. The combination of different hollow fiber types in these systems is possible. The systems are however difficult to manufacture and expensive and inflexible with regard to their use and are therefore hardly realized in practice (DE 4230194, U.S. Pat. No. 5,516,691). The arrangement as well as the materials of the employed hollow fibers are predetermined in all known systems.

Also known is a bioreactor that is assembled of modularly combinable elements containing fibers (DE 19932439). The elements comprise therein only one compartment, respectively, surrounding the exterior of the fibers. Supply or removal of fluids through the interior of the fibers is therefore excluded. The free flow of the liquid between the elements is not possible because they are separated by a semipermeable membrane. Use of the device according to DE 19932439 for filtration purposes is not proposed.

Known are also hollow fiber modules for filtration purposes that contain plates and fiber layers that are stacked on top one another (DE 2650341 and EP 350853). Between two plates each that are open at their center a hollow fiber layer is provided, respectively. The plates are inserted into a closed housing which contains a cavity surrounding the plates. The ends of the hollow fibers arranged between the individual plates are open to the exterior and project into the surrounding cavity. EP 454918 discloses a similar system with the difference that the hollow fibers are not clamped between plates but within a ring.

Individual supply or removal to and from the individual fiber layers is not possible as a result of the arrangement in the last mentioned systems (DE 19932439, EP 454918, and EP 350853). The use of these devices is limited to filtration. Disadvantageously, as a result of the additionally required housing, the application and variation possibilities are greatly limited.

Known is also a diffusion cell for ion exchange purposes according to DT 1642811 in which several layers of fiber mats and frames are stacked alternatingly. Because of the crossed arrangement of the hollow fibers a deformation of the hollow fibers at the crossing locations results. The hollow fibers are opened by cutting through the fiber layers after assembly wherein, as a systematic problem, a clean cut cannot be achieved but instead the fibers are frayed or even squeezed. The deformation and fraying of the fibers impairs the flow through the hollow fibers. It is also possible that particles will become detached from the frayed ends. As a result of the cut through the fiber layers undesirable dead space is produced in the diffusion cell favoring, inter alia, growth of bacteria. Directing flow individually to the diffusion cell is not possible according to DT 1642811.

The individual use of individual elements is not possible in connection with the known systems. The prior art systems are not designed for combination of different elements during fabrication or a modular combination of the elements by the user.

An adaptation in accordance with individual tasks of the user can be realized with the prior art systems only to a limited extent and expensively by means of individualized manufacture. The flow geometry and dimensions of the system cannot be changed.

It is an object of the present invention to provide a cassette that is versatile in regard to its application, in which cassette different fiber and hollow fiber materials can be employed individually or in combination, and which cassette enables as a module the assembly of a system.

According to the invention the object is solved by a fiber cassette
comprised of housing (1),
that is delimited by two congruent base surfaces (G) and at least one circumferential surface (M),
and contains in its interior at least one cavity,
comprising at least one layer of fibers or fiber bundles or hollow fibers (2),
that, in the interior of the housing (1), are arranged essentially parallel to at least one center plane and with their ends are anchored fixedly in the interior of the housing,
wherein a cavity forms an outer compartment (6),
that surrounds the exterior of the fibers (2),
wherein the center plane (E) does not intersect the base surfaces (G) within the cavity that forms the outer compartment (6),
wherein
the individual fibers (2) are arranged U-shaped or essentially parallel to one another and end within the interior of the housing,
and wherein the housing (1) has at least one opening for supplying and/or removing (9, 10, 11, 12, 13, 14) gases and/or liquids.

The fibers are arranged within the housing between the base surfaces. The arrangement of the fibers in this connection is parallel to one or several center planes between the base surfaces in contrast to the perpendicular arrangement relative to the base surface in conventional hollow fiber systems. The center plane is positioned usually parallel between the base surfaces or at least at an acute angle thereto. The center plane does not intersect the base surfaces within the housing or at least not within the cavity that forms the outer compartment. Advantageously, with this arrangement of the fibers relative to the center plane a flow direction results that is almost perpendicular to the fiber direction when connecting the outer compartments across the base surfaces.

The housing of the fiber cassette has preferably the shape of a body with polygonal or circular base surfaces, for example, a parallelepiped or cube or a cylinder. Preferred is a housing whose height (h) is small relative to its other dimensions. The term height (h) is to be understood as the average spacing between the base surfaces. By means of such a flat shape a large base surface results in comparison to the volume of the cassette. Advantageously, the cassettes can be stacked easily by means of their congruent large base surfaces.

A further advantage of the flat shape of the cassette is that it enables its use under a microscope when employing an appropriate light-transmitting material.

Preferably, the base surfaces (G) of the housing are congruent circles or polygons; especially preferred are regular polygons.

Advantageously, this shape of the base surface, when stacking several cassettes on top of one another, enables an arrangement of the fibers parallel to one another but also a staggered arrangement with different angles. For example, a square base surface enables an angle of 90°, 180°, 270°, or 360° between the fibers of the individual cassettes when arranging two cassettes above one another. A circular base surface enables accordingly an arrangement of the fibers at any angle.

Preferably, both base surfaces (G) extend parallel to one another and the circumferential surfaces are planar and perpendicular thereto. The housing in this case has the shape of a straight prism or cylinder.

In this case, the fibers are essentially perpendicular to the vertical line (h) and, accordingly, arranged parallel to the base surface (G) of the housing (1).

Possible is a configuration of the invention where the base surfaces (G) are not parallel but displaced at an angle relative to one another so that the assembly of several cassettes by means of their base surfaces (G) forms a body in the shape of a regular prism or a cylinder or a hollow cylinder. In the case of a cylinder, the individual cassette thus has the shape of a cylinder segment.

Depending on the application, fibers (2) of different shapes and materials are suitable and can be combined within one cassette.

Preferably, tubular hollow fibers are used. A cassette that contains hollow fibers is referred to in the following also as hollow fiber cassette. The hollow fibers are mounted fixedly with their ends in the interior of the cassette. The ends of the hollow fibers are open at one or both ends, respectively, or are sealed by being connected to the housing.

Preferably, the open ends of the hollow fibers are connected to at least one additional cavity in the housing. The interior of the hollow fibers and of the cavity or cavities connected thereto form thus an additional compartment that is referred to in the following as interior compartment (5).

The housing (1) forms a frame that surrounds the fibers (2) and the individual compartments (5, 6) in the interior of the body.

The inner (5) and outer (6) compartments according to the invention are separated by the hollow fiber material (2), the housing (1), and the attachment (3) of the hollow fibers in the housing in such a way that a substance exchange between these two compartments can be realized only through the material of the hollow fibers.

The attachment of the fibers in the housing of the cassette is realized preferably by embedding in a potting compound (3).

As a potting compound all conventional 1-component, 2-component, or 3-component adhesives (for example, epoxy resin, polyurethane) but also thermoplastic materials (for example, PE hotmelts) or reactive thermoplastic materials (for example, thermally processable polyurethane) or other curable liquid compounds (for example, liquid ceramic) can be used.

Advantageously, this is achieved in that on a support fibers are arranged in accordance with the required size parallel or in a U-shape relative to one another and the open ends are fused. Such hollow fiber mats with fibers that do not cross one another are inserted into a potting mold and the mold is fixed in a centrifuge. Under rotation, the potting compound is then introduced at the ends of the closed fiber ends. The rotation has the effect that only the fiber ends are embedded in the potting compound. In order for the interior of the hollow fibers to become accessible, the potting blocks after hardening of the potting compound are cut parallel to their longitudinal axis and perpendicularly to the hollow fibers so that a clean, smooth cut results. When the fiber ends are to remain closed, no cut is performed. The fiber units manufactured in this way are then glued into the housing.

Alternatively, the fibers can also be embedded directly into the material of the housing.

With the parallel or U-shaped arrangement of the fibers according to the invention, the entire fiber surface remains advantageously freely accessible and deformations of the fibers are prevented. By the exact cut of the fiber ends, smooth fiber ends result and dead space that cannot be flushed or flushed only with difficulty is prevented. Particularly the prevention of dead space is decisive for the use as a bioreactor because biological substances can deposit in dead spaces with minimal or no flow and decompose therein. Such deposition and decomposition contributes, on the one hand, to undesirable colonization of contaminants (bacteria, fungi) and, on the other hand, decomposition products and bacterial endotoxins are moved out by diffusion so that the growth of cells is negatively affected.

A further important advantage of the invention is the effective and exact cutting of the capillary ends. The smooth cut of the fiber ends embedded in the potting compound prevents fraying and deformation of the fiber ends. Because of the smooth fiber ends turbulence at the openings is prevented and uniform flow is ensured. When employing the fiber cassette as a bioreactor, uniform flow is a prerequisite for uniform nutrient supply of the cells that grow in the exterior space on the capillary membranes. The uniform nutrient supply is in turn a prerequisite for uniform growth and metabolism of the cells. In a bioreactor that is not uniformly supplied with nutrients, the cells would adjust their metabolism and their growth differently in accordance with the nutrient conditions in an undesirable way.

The cassette, depending on its configuration, can be used as a module within a system or can be used also individually. The housing (1) is preferably constructed such that it can be connected by gluing, plugging or fusing to the housings of other cassettes and the inner and/or outer compartments of several cassettes can be connected to one another without hose connections in a fluid-tight way.

The housing (1) contains at least one opening for supply and/or removal to and from the outer compartments. The opening can be a large surface area opening (13) in the upper or lower base surface (G) or in the circumferential surface that enables a direct connection to the outer compartment of other fiber cassettes.

Advantageously, the cassettes can be connected to one another with a large surface area by means of the congruent base surface. Cavities of additional compartments can be connected in accordance therewith by means of large surface area openings (14) in the base surface (G) or in a circumferential surface (M).

The housing contains in this connection means for a fixed or reversible fluid-tight connection of these openings (13,14) with those of other cassettes or at least one cover (4). The connection can be realized by gluing or fusing. Preferably, the connection is realized to be reversible by plug connections or clip connections with appropriate seals.

Alternatively, or as additional openings, the housing contains channels (7, 8, 9, 10, 11, 12) that function as supply and removal lines for fluids to the individual compartments. The connectors of the channels to the exterior are manufactured such that external connections, for example, hoses but also connections of solid material can be connected thereto. The connections can be manufactured by injection molding such that a simple opening action according to need and application is possible, even by the user. When individual openings are not needed, they can be closed by matching stops or caps or can remain closed.

The housing (1) and the cover (4) of the cassette are comprised preferably of a rigid or flexible polymer, composite material, glass, ceramic or metal.

As polymers all conventional plastic materials, for example, polyethylene, polypropylene, polyvinyl chloride, polyester, such as polycarbonate, polysulfones, polyethersulfones, but also silicones and biopolymers or composite materials can be used. A possible light-transmitting configuration of the cover or of the housing enables viewing in a microscope or other optical measurements.

As a cover (4), it is also possible to employ a membrane, made of a material such as silicone or another self-healing transparent polymer. Such a cover can be punctured by suitable instruments and can seal itself again. In this way, the manipulation of the cassette interior, even under a microscope, is enabled.

The cover (4) can also be comprised of a semipermeable flat membrane or a filter fabric with defined mesh width.

The cover (4) can also have openings for supply and/or removal of fluids to and from the inner and/or outer compartments.

The cover (4) can enclose one or several cavities that are respectively connected to one of the compartments of the cassette. Such a cover can be designed in the form of a tub (4′) but also in the form of an additional cassette (without fibers). The connection to the compartments of the cassette is realized by openings in the upper or lower base surface (G) or a circumferential surface (M).

In addition, it is possible to integrate cooling or heating devices, sensors, for example, optical and electrochemical sensors, ion-selective electrodes, into the housing or the cover of the cassette in order to measure parameters, such as temperature, pH value, O₂/CO₂ partial pressure, concentrations, conductivity, turbidity.

When arranging the fibers (2) in the cassette, they can be arranged to have a defined lateral spacing or they are arranged without order in a statistically defined package density (number of fibers per surface area). The arrangement of the fibers relative to one another is realized either in a U-shape or parallel.

In the parallel arrangement, the fibers are connected to the housing such that their two ends are positioned in different chambers at the opposite sides of the housing.

In the U-shaped arrangement, the two ends are positioned at the same side of the cassette. By mounting a partition at this cassette side, both ends can also be connected to two different chambers. A fixed connection to the housing is required for the U-shaped arrangement only at the side where the two ends of the fibers are located.

In the parallel arrangement as well as in the U-shaped arrangement, the hollow fibers can be open at one end (dead end module) or at both ends (flow through module) or can be closed at both ends.

The hollow fibers open at one end or at both ends enable, depending on the pore size, gas or liquid and/or substance exchange in accordance with the properties of the employed semipermeable membrane. Liquids can be pumped by connecting external pumps or pressure systems either with continuous pressure through the membrane or through the interior of the hollow fibers or can also be pumped with alternating pressure (push/pull method), for example, by connecting a syringe pump or piston pump.

Fibers that are closed at both ends and also filament fibers can be employed as filling threads, as a support material for adherent cells or microorganisms, as an absorption medium, and also for a substance-specific treatment of fluids.

Depending on the application, the fiber cassette contains one or several hundred fiber layers of a single or different fiber or total fiber materials.

The diameter of the fibers is, depending on the application, of a magnitude of a few μm to several millimeters.

The pore size of the hollow fibers, depending on the application, can range from a few nm up to μm in diameter. The use of closed-pore fibers is also possible.

Gas-permeable hollow fibers having a pore size within the nanometer range are used, for example, for oxygenation and closed-pore hollow fibers are used, for example, for the transfer of heat.

There are no limits with regard to the fiber or hollow fiber materials. The fibers are usually comprised of organic polymers, but inorganic materials, such as glass, ceramic, SiO₂, carbon or metal or mixtures thereof, are also possible. The materials can have hydrophilic or hydrophobic character. The polymers can be modified or unmodified or mixtures of these groups.

Possible biopolymers are, for example, cellulose, silk fibers and polymers produced by microorganisms and their derivatives, for example, cellulose esters and ethers.

Possible synthetic polymers are, for example, polyacrylonitrile, polyurethane, aliphatic and aromatic polyamides, polyimides, polysulfones, poly aryl ether sulfones, polycarbonates, polyolefins, for example, polyethylene, polypropylene, polvinylchloride, poly vinylidene difluoride, polytetrafluoroethylene, Teflon, poly phenylene oxide, poly benzimidazoles, poly benzimidazolones, poly benzoxazindiones, as well as their modifications or copolymers combined thereof, including graft copolymers or mixtures.

These polymers can have admixed thereto hydrophilic polymers, for example, polyethylene oxide, polyhydroxyether, polyethylene glycol, polyvinyl pyrrolidone, adsorption materials or other materials, for example, silicate, zeolites, active carbon, aluminum oxide.

In addition, fibers can also be filled with support materials, for example, active carbon or ion exchange resins.

An example for different materials in a hollow fiber cassette is the combination of microporous hollow fibers of polysulfone with active carbon fibers.

For the substance-specific treatment of fluids, functional groups or substances (in the following referred to as acceptors) are preferably immobilized on and/or in the fiber material of fibers or hollow fibers, closed or open at their ends; the functional groups or substances interact in a specific selective way with a substance contained in the fluid. Such interactions can be, for example, cation or and anion exchange, hydrophilic or hydrophobic interactions, hydrogen bonds, affinity or enzymatic or catalytic reactions. The acceptors can be, for example, antibodies or proteins or catalytically active substances, for example, enzymes or noble metals, complex compounds or non-ionic, ionic or zwitter ionic organic or inorganic substances or adsorbing substances. The term substance-specific treatment of a fluid refers to, for example, the catalysis of chemical reactions, the selective adsorption of substances or cells, but also the selective or non-specific prevention of said bonds. For adsorption, ion exchangers, immunoabsorbers, or hydrophobic acceptors can be used.

The substance-specific treatment however can also be the separation or retention of particles based on their size.

Advantageously, the hollow fiber cassettes according to the invention can be employed for different applications, for example, filtration, dialysis, osmosis, including reverse osmosis, separation, concentration of liquids, harvesting of cells, substances, antibodies or proteins, catalytic reaction of substances, adsorption or desorption of substances, enhancement of back filtration processes, gassing or degassing of media, physical transfer of heat, measurement of different parameters, such as pH value, temperatures, or the combination of two or several applications.

A further application of the fiber cassettes is the use as a bioreactor, for example, for culturing cells, bacteria and/or viruses. In this connection, they can grow in the interior of the fibers, in or on the fiber material or in suspension about the fibers.

A further aspect of the invention is a cassette system comprised of at least two fiber cassettes that are fixedly or reversibly connected to one another so as to be fluid-tight to the exterior. In this system, individual compartments (5, 6) of the individual cassettes are connected to one another. The connection is realized by means of an opening in the adjoining surfaces (13, 14) or by means of connecting channels (11, 12) preformed within the frame.

All possible applications of the individual cassettes can be combined and integrated as desired into a cassette system that is assembled by the user according to his requirements.

The connection of the cassettes to a system can be realized, for example, by fusing, gluing, or by a clip system or other means.

An important advantage of the invention is that a direct connection of the individual compartments of several cassettes is enabled without hose connections.

The fluid supply or fluid removal to and from the system can be realized by connectors in the cover or the housing. The cover can function as an additional fluid reservoir. If the individual compartments of the cassette are connected to one another, the medium supply to the individual cassettes can be realized by means of the connection to the neighboring cell.

The cassettes according to the invention can be combined in different arrangements with one another and can be connected either parallel or in series.

The configuration of the cassette system according to the invention and thus of the desired materials, depending on the configuration, can be carried out by the user as well as the manufacturer.

Advantageously, any number of cassettes can be combined. An additional housing surrounding the cassettes that would limit the versatility of the combinations is not required.

Preferably, the direct connection between the compartments (5, 6) of two cassettes is realized across most of the adjoining surfaces of the cassettes. Preferably, the contact is realized by a base surface (G), respectively, because they are congruent and are preferably large in relation to the circumferential surfaces. An additional advantage of the connection via the base surfaces is that a flow direction perpendicular to the fiber direction is realized. Because of this and because of the large-surface connection, an excellent substance and gas exchange between the connected compartments is realized.

Alternatively, the connection is also possible by means of one of the other surfaces or by a suitable arrangement of connecting channels pre-shaped in the housing.

A cassette system can be combined of cassettes (F) of different shapes.

Preferably, the cassettes have for this purpose parallel base surfaces (G) and the shape of a straight cylindrical or prism body, for example, a parallelepiped or square. In this case, the cassette system is comprised of vertically stacked cassettes that are connected to one another by means of their base surfaces (G).

When the cassettes have straight circumferential surfaces (M), for example, in the case of a prism shape, the cassette system can also have cassettes (F) connected laterally to one another by the circumferential surfaces (M).

The cassette system itself forms a cylindrical or prism body whose base surfaces are comprised of at least one base surface (G) of a single cassette (F).

In a special configuration of the invention the cassette system is comprised of cassettes (F) whose base surfaces (G) are not parallel to one another.

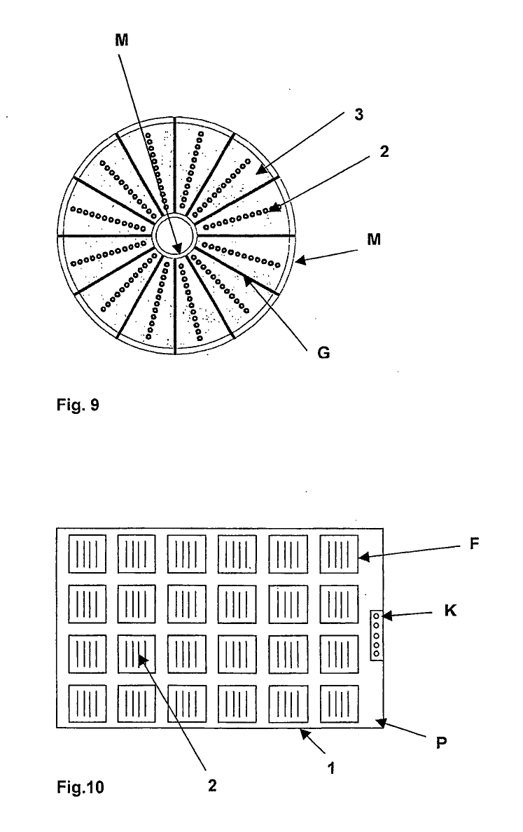

The cassettes are connected in a fan shape by the base surfaces (G) to one another. The cassette system forms preferably a regular prism, a cylinder or a hollow cylinder whose base surfaces are comprised of circumferential surfaces (M) of the individual cassettes (F). The cassettes either directly meet at the center of the cassette system or form at the center, in the case of a hollow cylinder, a tubular tunnel (see, for example, FIG. 9).

The medium supply is realized preferably through openings in the circumferential surfaces of the cassettes that form together the base surface of the prism or the cylinder. Alternatively, the media supply can also be realized through openings that open into the tubular tunnel in the center.

When the cassette system forms a regular circular cylinder, a rolling movement of the system can be achieved in that the system is placed on rollers. The cylinder rolls on its circumferential cylinder surface that is comprised of individual circumferential surfaces (M) of the individual cassettes. The latter system can be introduced into a cell culturing cabinet that is suitable for roller bottles (commercially available, for example, from Wheaton Science Products, N.J., U.S.A.).

When the system comprises a tubular opening at the center, an axle can be inserted therein that enables a rotation of the entire system.

Advantageously, in this way a rotation of the cylinder can be achieved without it having to be introduced into a cell culturing cabinet for roller bottles. Such a bioreactor can be operated with a heating device and fluid supply and fluid removal independent of a cell culturing cabinet.

Advantageously, by means of the continuous movement of such a system by rolling or rotation, a deposition of cells on the bottom is prevented and optimal flow of the media about the cells is achieved.

In numerous applications the subsequent use of many substance-specific treatments is desirable or necessary. Often, the purification of a protein, for example, requires several chromatography and filtration or dialysis steps in sequence. With the system according to the invention, this is greatly simplified by connecting in series cassettes of different materials. For example, in the first cassette a separation according to molecular size, in the second cassette by means of ion exchange material, and in the third cassette according to immunoaffinity can take place.

Advantageously, in the system according to the invention also an individual supply or removal of fluids to individual cassettes is possible. By using fibers of different materials (for example, different pore sizes, acceptor groups) in the individual cassettes, fractioning of the substances contained in the fluids is possible according to the interaction with the fiber materials (for example, exclusion by size, absorption).

The individual compartments of different cassettes can be separated from one another by the housing (1) or the cover (4).

Preferably, in such a fiber cassette system at least two adjoining compartments (5, 6) of different cassettes are either connected semipermeably to one another or separated from one another by a cover (4).

By selecting a cover (4) of appropriate material, the compartments can be also connected to one another semipermeably. This means that the cover can be a partition, a semipermeable membrane or a filter.

In a special configuration of the cassette system according to the invention, several cassettes together with a bottom plate (P) form a common housing that comprises channels for supply and/or removal of media to the individual cassettes. The connection to a housing can be realized, for example, by gluing or also by injection-molding a monolithic part. In this housing, compartments of different cassettes can be directly connected to a common compartment and/or channels in the carrier.

In such a plate-shaped cassette system cells can be cultured as well as examined in parallel in the individual cassettes. In this way, this cassette system is optimally suitable for use in mass examinations or similar applications.

The invention also includes an arrangement of at least one fiber cassette or at least one fiber cassette system and a carrier (T) that comprises for each fiber cassette or fiber cassette system devices for securing the cassette or the cassette system and devices for the supply and/or removal of media to the individual cassettes. The arrangement of several fiber cassettes or cassette systems in the carrier is realized horizontally adjacent to one another and/or stacked above one another. The connection of the individual cassettes to the carrier can be realized by a base surface or a circumferential surface. The carriers have the function of geometric fixation of the individual systems as well as providing supply and removal of media and products to and from the cassettes through the channels or hoses provided in the carrier.

The cassettes, depending on the medium and application, can be supplied by the supply channels/hoses individually, in series or parallel. The cassettes/cassette systems can be connected fixedly or reversibly to the carrier. A flexible connection is achieved, for example, by plug and/or clip connections.

The devices for securing are preferably in the form of insertion slots or slides into which the cassettes can be reversibly inserted.

Alternatively, the securing action is realized by special connectors that are inserted into recesses in a plate-shaped carrier. In their interior, these connectors comprise preferably channels that are connected as supply and removal lines to the individual compartments in the cassettes. By insertion of the connectors, the channels in the connectors are reversibly and in a sterile fashion connected to the channels in the carrier for media supply and media removal.

A preferred configuration of the carrier is in the form of a rack in which several cassettes are vertically stacked or placed adjacent to one another in a space-saving way. Another preferred configuration of the carrier is a plate on which several cassettes are arranged horizontally adjacent to one another.

With such an arrangement on a carrier, a system of cassettes/cassette systems is obtained that is optimally suitable for processing in connection with mass examinations or similar applications.

The horizontal arrangement of several cassettes on a carrier in the form of a plate enables an excellent access for manipulation and examination of individual cassettes/cassette systems without the connection to the plate having to be released.

In an arrangement in a carrier in the form of a rack, individual cassettes can be removed for examination or manipulation.

Automation by use of robot arms is possible.

Based on the attached drawings, embodiments of the invention will be explained in detail. It is shown in:

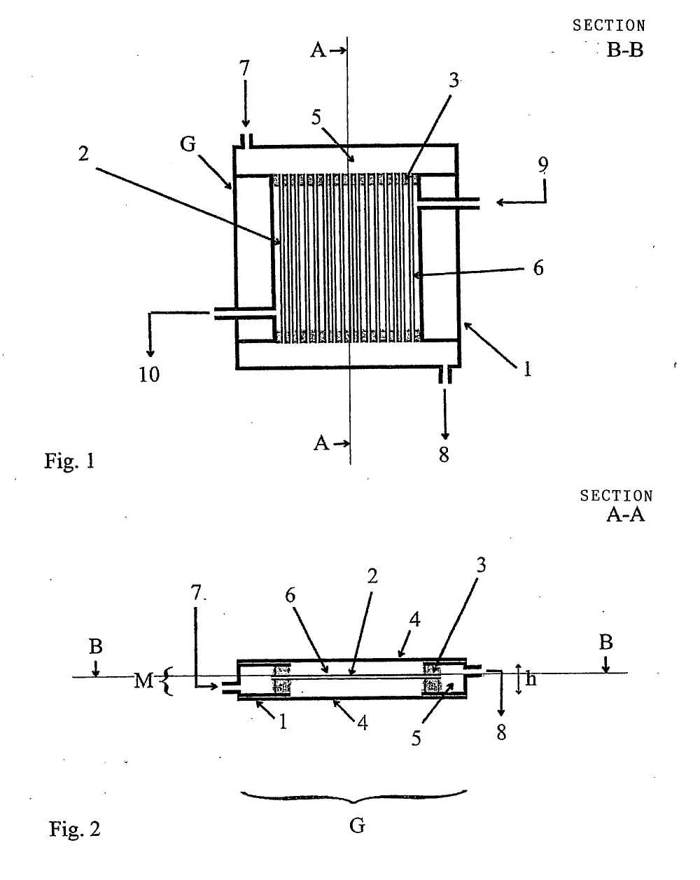

FIG. 1 a horizontal section of a hollow fiber cassette with open ends of the hollow fibers;

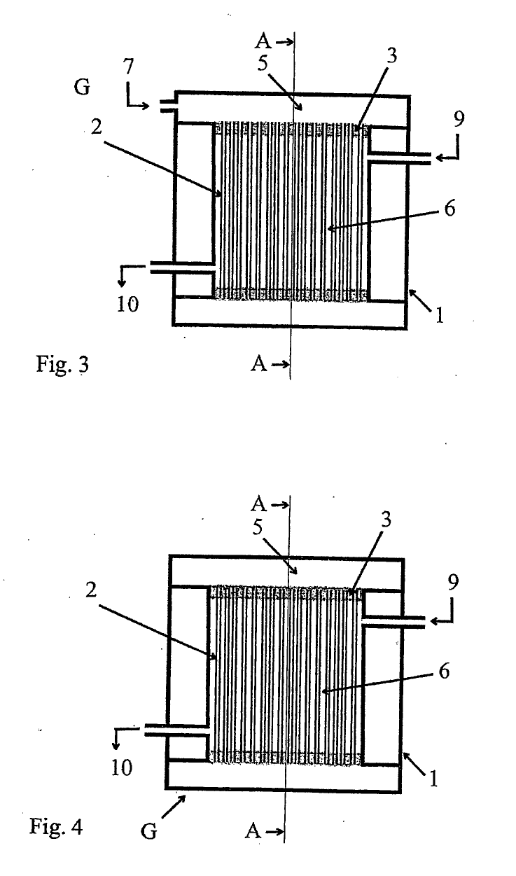

FIG. 2 a vertical section of a hollow fiber cassette according to FIG. 1;

FIG. 3 a horizontal section of a hollow fiber cassette in which the hollow fibers are open at one end and closed at the other;

FIG. 4 a horizontal section of a hollow fiber cassette with closed ends of the hollow fibers;

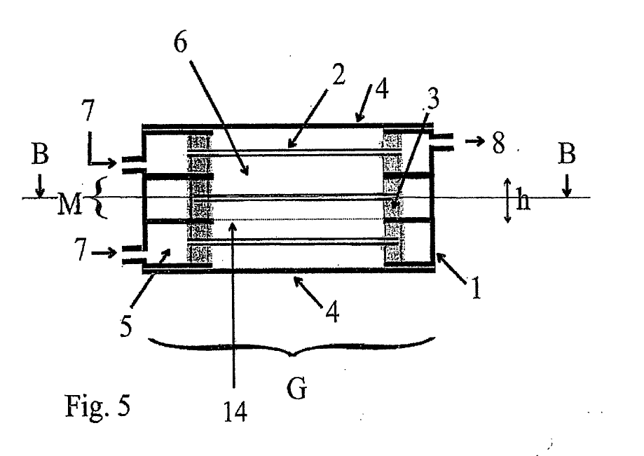

FIG. 5 a section illustration of a hollow fiber cassette system with 3 different stacked fiber cassettes;

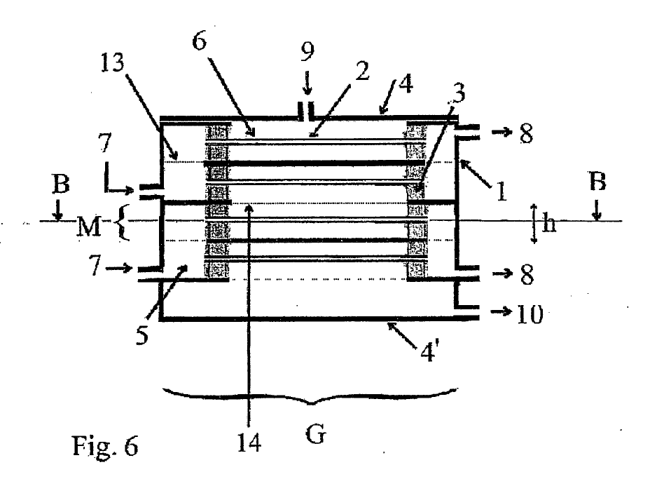

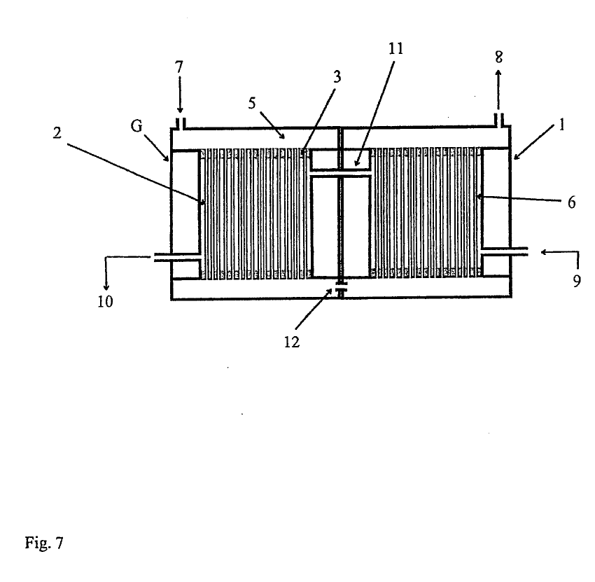

FIG. 6 a section view of a hollow fiber cassette system with 4 different cassettes containing fibers;

FIG. 7 a section view of a hollow fiber cassette system with 2 fiber cassettes arranged adjacent to one another;

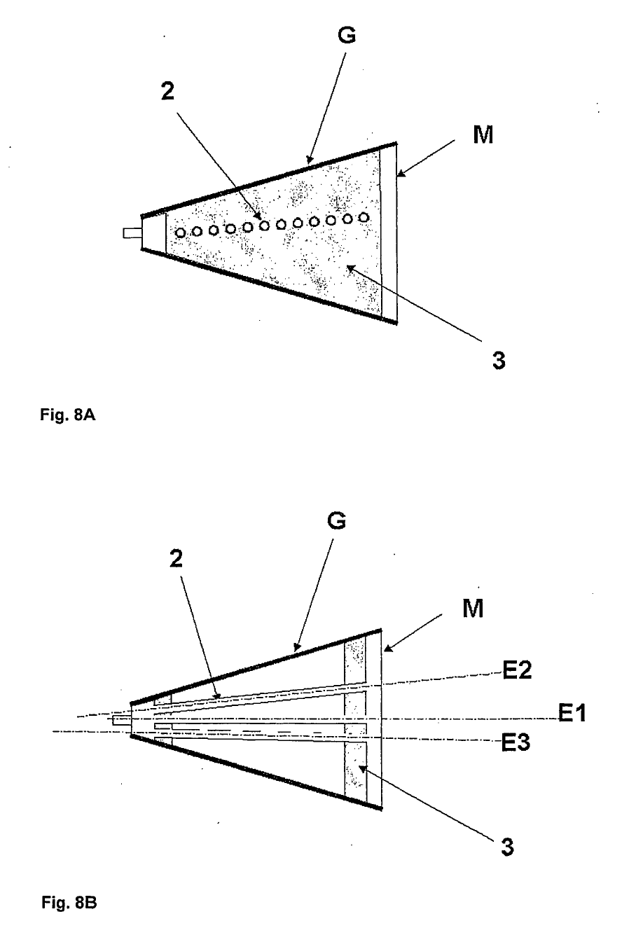

FIG. 8 a vertical section of two hollow fiber cassettes with base surfaces that are arranged staggered at an angle relative to one another;

FIG. 9 a section view of a cassette system for culturing cells while being rotated;

FIG. 10 a plan view onto a fiber cassette system comprised of 24 hollow fiber cassettes and a base plate;

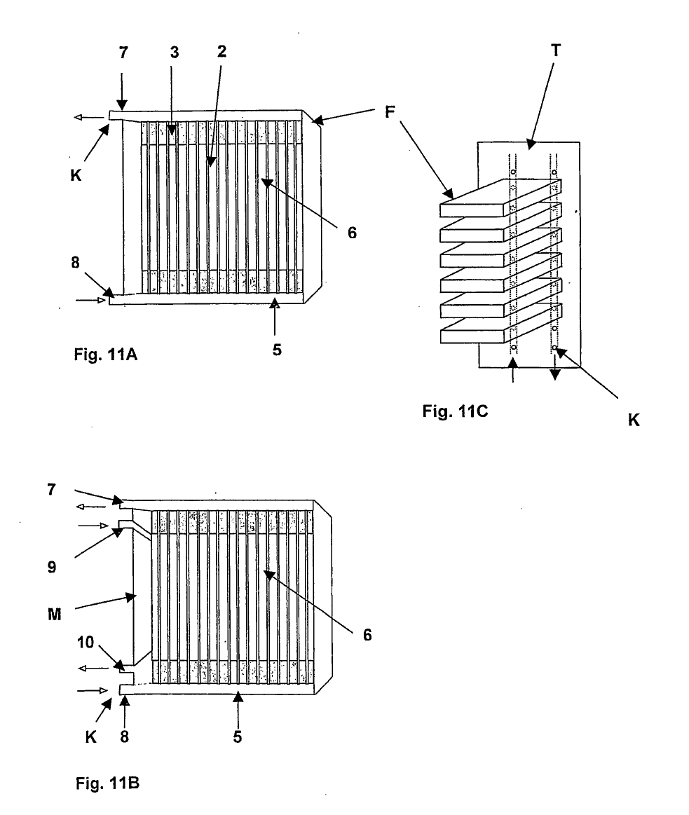

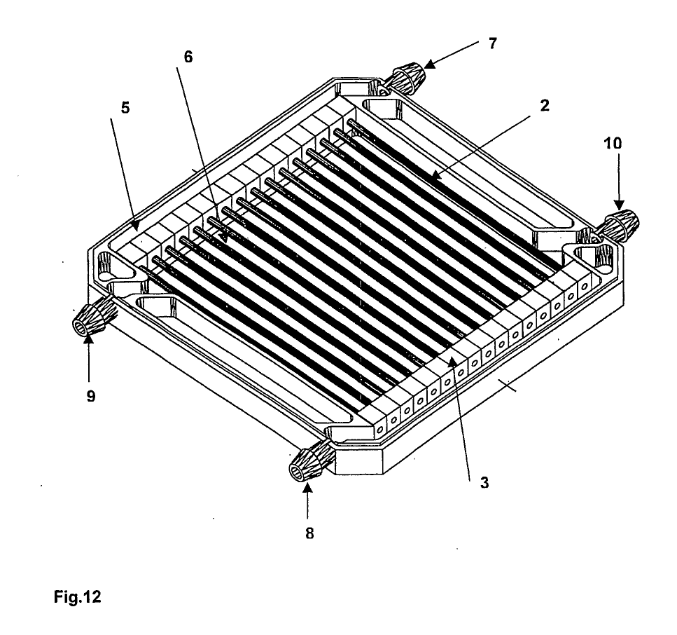

FIG. 11 a three-dimensional illustration of an arrangement comprised of a rack-type carrier and 6 hollow fiber cassettes inserted therein, as well as a horizontal section of two hollow fiber cassettes with connectors for connecting to a carrier;

FIG. 12 a three-dimensional illustration of a hollow fiber cassette system analog to FIGS. 1 and 2;

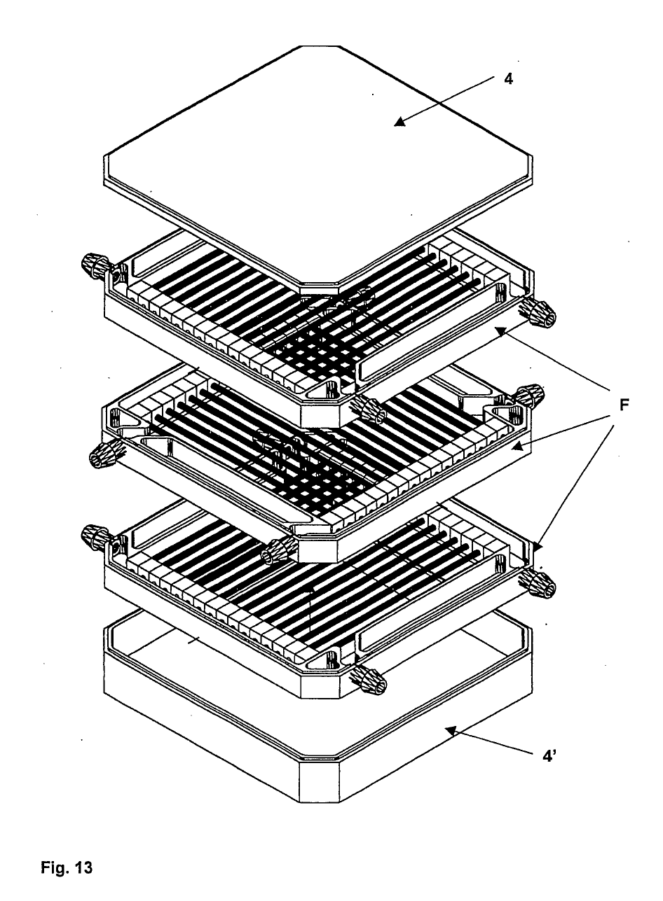

FIG. 13 a three-dimensional illustration of a hollow fiber cassette system with 3 cassettes as in FIG. 12;

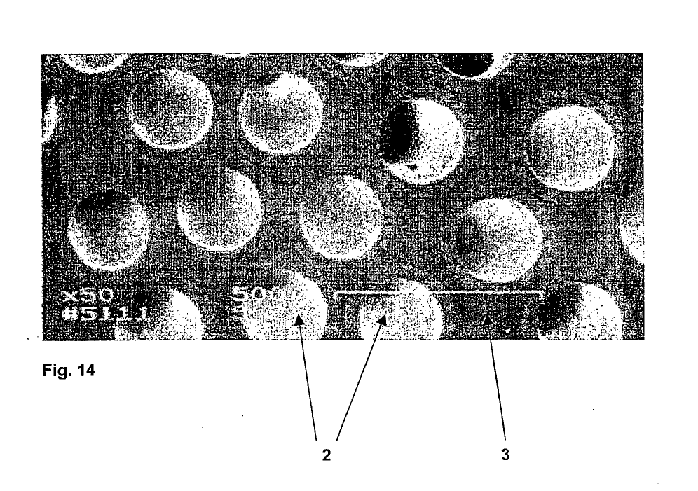

FIG. 14 an image of a section of a potting compound with open fiber ends.

FIG. 1 shows a horizontal section of a hollow fiber cassette with two parallel square base surfaces G having two hollow fiber ends (2) that are open.

In a housing (1) a plane layer of parallel arranged hollow fibers (2) is arranged. The hollow fibers (2) are embedded at their ends in the potting compound (3) within the housing (1) so that both open ends of the hollow fibers (2) point toward the inner compartment (5). For this purpose, a polysulfone ultrafiltration hollow fiber made by company Ascalon GmbH, Bergieβhubel, Germany (280 μm outer diameter) is wound in parallel about a 60 mm wide metal plate. After the fibers have been wound in a single layer with a width of 5 cm, the wound fiber is fixed at the front and the back on both edges of the metal plate with a narrow adhesive tape (1 mm), respectively, that extends perpendicularly to the hollow fibers. The hollow fibers are then cut open at both edges of the metal plate with a knife. In this way, two fiber mats are obtained in which the hollow fibers (2) that are open at both ends are held together by two adhesive tapes. The open ends of the hollow fibers (2) are fused by means of a bar fusing device. The mats are then placed into a potting mold and secured. The potting mold is secured in a centrifuge and a statically mixed two-component adhesive polyurethane comprised of polyol and polyisocyanate made by Morton is applied as a potting compound (3) under rotation (600 revolutions per minute). After 30 minutes the mold is removed from the centrifuge. After an additional hour, the hollow fiber mat that at the ends is 5 mm rectangular is removed from the mold. A polyurethane potting block (3) has the dimensions of 45 mm×5 mm×3 mm. After approximately 12 hours of post-curing, the polyurethane blocks (3) are cut parallel to their longitudinal axis and perpendicularly to the hollow fibers (2) so that the interior of the hollow fibers is accessible and, as shown in FIG. 4, a clean smooth cut results. Because of the supporting action of the potting compound the hollow fibers do not fray and are opened cleanly and smoothly without dead spaces. The completed fiber unit is glued by means of polyurethane into the housing (1).

The housing (1) is divided by its construction, by the potting compound (3) and the hollow fibers (2) into the inner compartment (5) and the outer compartment (6). A substance exchange between the compartments can be realized exclusively by the pores of the hollow fibers.

The housing (1) contains the channels (7 and 8) for supply and removal of gases and liquids to and from the inner compartments (5), and the corresponding channels (9 and 10) for supply and removal of gases and liquids to and from the outer compartments (6).

FIG. 2 shows a vertical section of the hollow fiber cassette according to FIG. 1. The outer compartment (6) is closed in the upward and downward directions by a cover (4) on the base surfaces (G), respectively. The covers are configured as flat lids which span across the entire base surface, respectively. The fluid-tight connection of the covers to the housing is realized by plug connections, not shown in the illustration, in the base surfaces. Illustrated are the supply and removal lines (7, 8) for compartment (5) in the circumferential surfaces (M). Not illustrated are the supply and removal lines (9, 10) for compartment (6) in the circumferential surfaces on the end face and back of the cassette.

The hollow fiber cassette according to FIG. 1 and FIG. 2 can be used, for example, for dialysis. For this application, a semipermeable hollow fiber having an exclusion size in the range of 2-50 kD (50% cutoff) is selected, for example. In this connection, the liquid to be dialyzed is guided via the supply and removal lines (7, 8) through the inner compartment (5) of the hollow fiber (2).

The buffer solution relative to which the dialysis is to be performed is supplied through the supply and removal lines (9, 10) and flows about the outer side of the hollow fibers (2) in the compartment (6).

The hollow fiber cassette illustrated in FIG. 1 and FIG. 2 can also be used as a bioreactor that can be used in connection with a microscope. The upper and lower covers (4) are comprised in this connection of a material that is suitable for optical microscopy. In this connection, cells or microorganisms grow in the outer compartment (6) either by adhesion or in suspension. The supply with nutrient media, oxygen, and carbon dioxide is realized advantageously by a hose system connected to the supply and removal lines (7, 8).

Supply and removal lines (9, 10) are used, for example, for introducing and harvesting the cells or the microorganisms. For following the growth and morphology of the cells and other parameters that can be optically evaluated, the hollow fiber cassette is placed under a microscope.

The hollow fiber cassette illustrated in FIG. 1 and FIG. 2 can also be used as a multi-phase reactor for extraction. In this connection, two different media (A and B) are introduced into the inner and outer compartments. For example, through the openings (7) and (8) an aqueous medium A that contains extracting substances is flown through the interior of the hollow fibers (2). An organic medium as medium B flows about the exterior of the hollow fibers, for example. The flavoring agents are better soluble in the organic medium B and pass through the hollow fibers into the medium B. By the separation of the media an effective extraction is possible. Such an extraction can be used, for example, for purifying flavoring substances.

FIG. 3 shows a horizontal section of a hollow fiber cassette in which the hollow fiber are open only at one end.

The device is analog to that described in connection with FIG. 1 with the difference that the hollow fibers are open only at one side. Accordingly, the inner compartment (5) has only one connector (7) through which fluids can be introduced into and removed from the interior of the hollow fiber (2).

The hollow fiber cassette according to FIG. 3 can be used, for example, for gassing media. For this purpose, a gas is introduced through connector (7) through the hollow fibers.

The hollow fiber cassette according to FIG. 3 can also be used for filtration. For this purpose, advantageously the liquid to be filtered is supplied through the supply and removal lines (9, 10) into the outer compartment and the filtered liquid is removed through the connector (7).

The hollow fiber cassette according to FIG. 3 can also be used, for example, for selective bonding or reaction of substances. In this connection, in or on the hollow fibers (2) corresponding substance-specific bonding or catalytically acting groups are provided. The solution that contains the substance to be reacted is advantageously introduced through the supply and removal lines (9, 10) into the outer compartment and the reacted liquid is removed through the connector 7.

FIG. 4 shows a horizontal section of a hollow fiber cassette in which the two hollow fiber ends are closed.

The device is analog to that described in FIG. 1 with the difference that the hollow fibers are closed at both ends. The hollow fiber cassette according to FIG. 4 can be used, for example, for cultivating adherent cells. The cells grow on the exterior side of the hollow fibers. The supply with nutrients and oxygen and carbon dioxide is realized either through the openings (9, 10) or advantageously by means of combination with other cassettes in analogy to FIG. 5.

FIG. 5 shows a vertical projection through a hollow fiber cassette system.

In this system, three different hollow fiber cassettes, one cassette each constructed in analogy to FIGS. 3, 4, and 1, are connected to one another by plug connections that are provided in the base surfaces (G) and are not illustrated in the drawing. The cavities of the individual cassettes that surround the fibers are connected to one another across large surface areas via openings (14, illustrated by a dashed line) in the base surfaces and form a common outer compartment (6). Covers (4) that are connected by plug connections to the base surfaces enclose the common outer compartment (6) in a fluid-tight way in the upward and downward direction.

Since the cassette system illustrated in FIG. 5 has only one common outer compartment (6), one supply line and one removal line (9, 10) for the common outer compartment are sufficient. They are located in the end face or back of the cassette and not illustrated.

The system according to FIG. 5 is an example of a possible bioreactor in which adherent cells grow in the central cassette (analog to FIG. 4) on the hollow fibers closed at both ends or on carrier fibers and are supplied through the other two hollow fiber cassettes with nutrients (upper cassette analog to FIG. 1), oxygen and carbon dioxide (lower cassette analog to FIG. 3).

The cells can be supplied not only with media through the hollow fibers: also, products secreted by the cells into the medium, for example, antibodies, can be separated from the cells and purified.

The production of a protein by means of cell culture and simultaneous purification by several substance-specific separation steps is explained in an exemplary fashion in FIG. 6. The system illustrated in FIG. 6 is comprised of four stacked hollow fiber cassettes connected across large surface areas and closed by two covers (4, 4′) at the top and the bottom. The individual cassettes are connected to one another and to the covers by plug connections, not illustrated, in the base surfaces in a fluid-tight way. The upper cover (4) has a connector (9) for supply of media to the outer compartment (6) of the uppermost cassette. The inner compartments of the first and the second cassettes are connected to one another by openings (13, illustrated by a dotted line) in the base surfaces across a large surface area. In the same way, the inner compartments of the third and the fourth cassettes are connected to one another. The outer compartments of the second and third cassettes are connected in analogy by corresponding openings in the base surfaces (14, illustrated by dotted line).

The lower cover (4′) comprises a cavity acting as a collecting container and connected to the outer compartment of the lowermost cassette and also comprises a connector (10) connected to the cavity.

In the outer compartment (6) of the uppermost cassette the cells grow and secrete the desired protein into the medium. The medium is supplied through connector (9) in the upper cover (4) to the outer compartment of the uppermost cassette and flows through the entire system downwardly and is removed through connector (10) in the lower cover (4′). The connectors (9) and (10) can be connected by a pump in order to ensure a continuous media circulation.

By means of the hollow fibers (2) with large pore size acting as a pre-filter the medium containing the protein is separated in a first filtration step from suspended particles, cells, and cell residues and guided through the connection of the two inner compartments into the second cassette. Here the protein is separated from the protein solution by hollow fibers of a smaller pore size from the larger molecules and passes through the fiber material into the outer compartment of the second cassette that is connected to the outer compartment of the third cassette. The inner compartment of the second cassette can be flushed optionally via the connectors (7) and (8).

By means of the hollow fibers containing acceptor groups in the third cassette other undesirable substances are separated from the protein by affinity chromatography. The solution containing the desired protein passes through the fiber material into the inner compartment of the third cassette which is connected to the inner compartment of the lowermost cassette. The undesirable substances remain within the outer compartment of the third cassette and can optionally also be removed by an additional connector (not illustrated in the section view) of the outer compartment.

By means of the pore size of the hollow fibers of the lower cassette in the nanometer range the protein in the inner compartment is concentrated and can be removed through the connectors (7) and (8). The liquid of the medium and smaller molecules flow through the hollow fibers into the outer compartment of the lowermost chamber and into the collecting container connected thereto and provided within the cover (4). The collecting container can be emptied through connector (10).

Advantageously, for renewal of the cells or when the hollow fibers in the upper cassettes are clogged, the respective cassettes can be individually exchanged. The lower cassettes whose inner compartments contain the concentrated protein solution can remain in use. In this way, a possible loss of protein by adsorption on surfaces is minimized.

In order to enlarge the system, in place of the uppermost cassette, several of the same type, connected with their inner compartments, can be stacked on top one another, for example.

Heating wires or closed-pore hollow fibers through which water heated to the desired temperature can be passed can be inserted into the uppermost cassette. By means of such a heating device culturing outside of a special culturing cabinet is made possible. At the same time, by means of an appropriate cooling device in the lower two cassettes the concentrated protein solution can be cooled.

FIG. 7 shows a horizontal projection of a hollow fiber cassette system.

In this system, two hollow fiber cassettes, configured in analogy to FIG. 1 or FIG. 2, respectively, are connected to one another. The cassettes are connected to one another by plug connections (not illustrated) in their circumferential surfaces (M) and connecting channels (11, 12) between the inner (5) and outer (6) compartments.

For the system illustrated in FIG. 7, the same fields of application as for the fiber cassettes of FIGS. 1 and 2 are possible wherein by serial connection of two cassettes the volume of the system is increased.

FIGS. 8A and 8B show a vertical section of a hollow fiber cassette that is configured in analogy to FIG. 1 and FIG. 2, with the difference that rectangular base surfaces are displaced at an angle relative to one another. In both Figures, the fibers (2) are practically parallel to one of the base surfaces (G) or an intermediate center plane (E). The orientation of the fibers in FIG. 8A is perpendicular to the fiber orientation in FIG. 8B. Both cassettes contain connectors for fluid supply and removal to and from the individual compartments; they are not illustrated in FIG. 8A and FIG. 8B.

In FIG. 8A a hollow fiber layer (2) is arranged that is illustrated by small circles. The interior of the circles represents the fiber cavity. The fibers are arranged parallel to the right and left circumferential surfaces (M).

In FIG. 8B three fiber layers (2) are illustrated that are perpendicular or almost perpendicular to the right and left circumferential surfaces (M). The fibers are positioned in a central plane (E1, E2, E3), respectively, between the base surfaces (G) of the cassette.

FIG. 9 shows a three-dimensional illustration of a cassette system for growing cells under rolling conditions. This system is comprised of 12 cassettes (F) configured as illustrated in FIG. 8A, with the difference that one circumferential surface (M) is concavely curved and the other is convexly curved. The individual fiber cassettes are comprised or assembled such that the cassette system forms a cylinder with circular base surface. The individual cassettes are connected by a base surface (G), respectively, by a plug system across a large surface areas.

The convex circumferential surfaces of the individual cassettes face outwardly and form the circumferential surface of the cylinder. The concave circumferential surfaces of the individual cassettes face inwardly and form at the center of the cylinder a cavity through which a structural component (not illustrated in FIG. 9) can be inserted.

The structural component comprises supply and removal lines that can be connected to the supply and removal lines of the individual compartments of the cassettes (5, 6). The supply and removal lines are not illustrated in FIG. 9. The structural component acts simultaneously as an axis of rotation and can be configured such that by connecting an electric motor at one end a rotation of the cylinder can be achieved.

Such a bio reactor can be operated independently of a cell culturing cabinet with a heating device and with a supply and removal device as described in connection with FIG. 6.

FIG. 10 shows a plan view onto a fiber cassettes comprised of 24 (4×6) hollow fiber cassettes (F) and a base plate (P) for use as a bioreactor for cellular mass examinations. A fixed connection of the cassettes to the base plate is achieved in that the base plate (P) and the individual cassettes (F) are formed as a unitary part by injection molding. The individual cassettes and the base plate form a common housing (1) that in its interior, not illustrated in FIG. 10, contains channels and at the side connectors (K) for the supply and removal of fluids.

The individual cassettes are constructed in analogy to FIG. 1 with the difference that the channels in the interior of the bottom plate are connected directly to the interior of the hollow fibers and form together with the cavities of the hollow fibers a common inner compartment for all cassettes and the outer compartments have no separate supply lines.

The individual compartments and the potting compound are not illustrated in FIG. 10 for clarity of the drawings; the hollow fibers are illustrated only schematically by dashed lines.

Each cassette (F) comprises its own outer compartment separated from the other cassettes and surrounding the hollow fibers (2). The outer compartments are closed by a base plate (P) in the downward direction but are open in the upward direction across a large surface area. The openings of the outer compartments can be covered in the upward direction by individual lids covering one compartment each or by a continuous cover that covers the entire upper surface of the housing.

In such cassette system cells can be introduced through the openings of the outer compartments in the upward direction and grow, depending on the cell type, in, on or in the suspension about the fibers (2). This form of cassette system in analogy to a multi-well plate (as used, for example, frequently for immuno assays) enables the examination of cells that grow parallel in the individual cassettes (F). For simplifying the illustration, the cassette system is illustrated with 24 cassettes; a corresponding embodiment with, for example, 96 (8×12) cassettes is possible also. The complete plate can therefore also be used in an automated operation, for example, in a plate reading device or examined under a microscope.

Such a fiber cassette system can be used, for example, for the patient-specific screening in medicine for chemotherapy. In this connection, patient-own cells, i.e., tumor cells, can be tested in the individual fiber cassettes with regard to reaction relative to different chemotherapy agents. The results of such tests enable the planning of a more effective treatment concept and a therapy that is matched individually to the patient.

In FIG. 11A and FIG. 11B cassettes are illustrated that can be connected by special connectors (K) to a carrier (T). In FIG. 11C, such an arrangement comprised of a carrier (T) and 6 cassettes (F) is illustrated.

The cassettes illustrated in FIGS. 11A and FIG. 11B are constructed in analogy to the cassette of FIG. 1 and FIG. 2 with the difference that the supply and removal lines (7, 8, 9, 10) are all located within a circumferential surface (M) and are configured in the form of connectors (K) that enable the connection to a carrier (T). The cassette illustrated in FIG. 11A comprises exclusively supply and removal lines (7, 8) to the compartment (5). The cassette illustrated in FIG. 11B comprises in addition also supply and removal lines (9, 10) to the outer compartment (6). The media flow direction through the cassette is illustrated by arrows, respectively.

In FIG. 11C, an arrangement comprised of a plate-shaped carrier (T) functioning as a rack with 9 insertion slots is shown. In 6 of the insertion slots hollow fiber cassettes (F) are inserted that are constructed in analogy to those illustrated in FIG. 11A. The uppermost and the two lowermost insertion slots remain free. The carrier (T) comprises in the interior channels for supply and removal of media, indicated by dashed double lines, and recesses (A), indicated by circles, for connection to the connectors (K) of the cassettes. The media flow direction for the channels in the carrier is illustrated by arrows.

The connection of the connectors (K) with the recesses (A) serve for reversible sterile connection of the channels in the carrier with the compartments (5, 6) in the cassette (F). At the same time, this connection also serves for fixation of the individual cassettes (F) in the carrier (T).

An application of this arrangement is culturing of cells in the individual fiber cassettes functioning as bioreactors. Advantageously, this arrangement enables a space-saving arrangement of a plurality of bioreactors and the removal of individual cassettes, for example, by robot arms.

In analogy, by means of connectors or in slides of an appropriate rack-shaped carrier entire hollow fiber cassettes systems can be secured, for example, those illustrated in FIG. 10.

FIG. 12 shows a three-dimensional illustration of a hollow fiber cassette, configured as shown in FIGS. 1 and 2 with the difference that the supply and removal lines (9, 10) to the inner and outer compartments are not distributed across 4 but only 2 circumferential surfaces and the base surfaces are cut off at their corners.

FIG. 13 shows a three-dimensional illustration of a hollow fiber cassettes system comprised of 3 cassettes (F) in accordance with FIG. 12 and a flat cover (4) at the top as well as a tub-shaped cover (4′) at the bottom. The cassettes in this illustration are stacked such that a vertically displaced fiber orientation results.

FIG. 14 shows an image taken by light microscope of open hollow fiber ends (2) embedded in the potting compound (3).

List of Employed Reference Numerals:

In the illustrated drawings the individual elements of the hollow fiber cassette are numbered as follows:
1 housing
2 hollow fibers
3 potting compound
4 cover
5 inner compartment, connected to the interior of the hollow fibers
6 outer compartment, connected to the exterior of the hollow fibers
7 and 8 supply and removal lines to the inner compartment 5 9 and 10 supply and removal lines to the outer compartment 6
11 connecting channel between the compartments of two cassettes 12 connecting channel between the outer compartments of two cassettes
13 large surface area opening in the base surface to the outer compartment
14 large surface area opening in the base surface to the inner compartment
A recess in a carrier for connecting to a connector
E1, E2, E3 center planes
F individual fiber cassette in a system or in an arrangement
G base surface
h vertical line
K connectors
M circumferential surface
P base plate
T carrier

What is claimed is:

1-17. (canceled)

18. A fiber cassette comprising:
a housing that is delimited by two congruent base surfaces and at least one circumferential surface and has an interior having at least one cavity;
at least one layer of fibers arranged in the interior of the housing essentially parallel to at least one center plane of the housing, wherein ends of the fibers are anchored fixedly in the interior of the housing;
wherein a first one of the at least one cavity defines an outer compartment that surrounds the fibers externally;
wherein the at least one center plane does not intersect the base surfaces within the outer compartment;
wherein the fibers are arranged U-shaped or essentially parallel to one another and end within the interior of the housing;
wherein the housing has at least one opening for supplying and/or removing fluids.

19. The fiber cassette according to claim 18, wherein the fibers are arranged in fiber bundles.

20. The fiber cassette according to claim 18, wherein the fibers are hollow fibers.

21. The fiber cassette according to claim 18, wherein the base surfaces of the housing are polygonal or circular.

22. The fiber cassette according to claim 18, wherein the fibers are comprised of different fiber materials and wherein the fibers comprised of different fiber materials are combined with one another in the fiber cassette.

23. The fiber cassette according to 18, wherein at least one part of the fibers are hollow fibers or hollow fiber membranes.

24. The fiber cassette according to claim 23, wherein the hollow fibers have open ends at least at one end of the hollow fibers and wherein a second one of the at least one cavity defines at least one additional inner compartment wherein the open ends of the hollow fibers are connected to the at least one additional inner compartment so that a substance exchange between the at least one inner compartment and the outer compartment is possible only through a material of the hollow fibers.

25. The fiber cassette according to claim 18, wherein the housing comprises at least one large surface area opening that opens into the at least one cavity arranged in the interior, wherein the at least one large surface area opening comprises means for providing a fluid-tight connection to cavities of an additional fiber cassette or one or more covers to be connected to the fiber cassette.

26. The fiber cassette according to claim 18, wherein the housing has channels for supplying or removing fluids to and from the at least one cavity.

27. The fiber cassette according to claim 18, further comprising at least one cover connected to the housing, wherein at least one of the housing and the at least one cover is comprised of a material selected from the group consisting of flexible polymer, rigid polymer, metal, glass, ceramic, and a light-transmitting material.

28. The fiber cassette according to claim 18, further comprising at least one cover connected to the housing, wherein the at least one cover is comprised of a material that, after having been punctured, seals itself or is comprised of a semipermeable membrane or a filter fabric.

29. The fiber cassette according to claim 18, further comprising at least one cover connected to the housing, wherein the at least one cover has at least one opening for supplying and/or removing fluids.

30. The fiber cassette according to claim 29, wherein the at least one cover has at least one cavity that is connected to the at least one cavity of the housing.

31. The fiber cassette according to claim 18, further comprising reagents immobilized on and/or in material of the fibers.

32. The fibercassette according to claim 31, wherein the reagents perform a task selected from the group consisting of binding substances or cells of the fluid and preventing a binding action of substances or cells of the fluid or are selected from the group consisting of chemical catalysts, biochemical catalysts, proteins, nucleic acids, complex compounds, and noble metals.

33. The fiber cassette according to claim 18, containing particles that enable a substance-specific treatment of a fluid.

34. A fiber cassette system comprised of at least two fiber cassettes that are fluid-tightly and fixedly or releasably connected to one another, wherein compartments of the at least two fiber cassettes are connected to one another across most of adjoining surfaces of the at least two fiber cassettes or by an arrangement of pre-shaped connecting channels of the at least two fiber cassettes.

35. The fiber cassette system according to claim 34, wherein at least two of the compartments of different cassettes are semipermeably connected to one another or are separated from one another by a cover.

36. The fiber cassette system according to claim 34, further comprising a bottom plate, wherein at least two fiber cassettes form together with the bottom plate a common housing that comprises channels for supplying and/or removing media to the individual fiber cassettes.

37. An arrangement of at least one fiber cassette according to claim 18 or a fiber cassette system according to claim 34 and a carrier, wherein the carrier has, for each fiber cassette or each fiber cassette system, devices for securing and for supplying and/or removing media to and from the individual cassettes.
